(19) United States
(12) Patent Application Publication
(10) Pub. No.: US 2015/0328138 A1
(43) Pub. Date: Nov. 19, 2015

(54) HAIR CLUE

(71) Applicants: Mei-Ling LO, Taipei City (TW); Kuo-Ching CHIANG, New Taipei City (TW)

(72) Inventors: Mei-Ling LO, Taipei City (TW); Kuo-Ching CHIANG, New Taipei City (TW)

(21) Appl. No.: 14/813,129

(22) Filed: Jul. 30, 2015

(30) Foreign Application Priority Data
Jan. 8, 2014 (TW) 103126383

(51) Int. Cl. A61K 8/97; A61K 8/34; A61Q 5/06

(52) U.S. Cl. CPC A61K 8/97; A61Q 5/065; A61Q 5/06; A61K 8/34; A61K 2800/438; A61K 2800/592; A61K 2800/52

(57) ABSTRACT

A hair glue or spray includes gel, solvent, and photochromic or thermochromic material doped therein to allow hair to change color.

### CROSS-REFERENCE TO RELATED APPLICATION

This application claims the benefit of TAIWAN Patent Application No. 103126383, filed Aug. 1, 2014.

### TECHNICAL FIELD

The present invention relates to hair glue, and particularly to a hair glue composition.

### BACKGROUND OF RELATED ART

Modern people usually spray or smear hair glue on their hair to make hair styles. The present invention provides a new hair glue.

### SUMMARY

A hair glue includes gel, solvent, and photochromic or thermochromic material doped therein to allow hair to change color. The hair glue includes temperature control agent, light stabilizer, ultraviolet (UV) absorber or the combination thereof. The solvent includes alcohol. The alcohol includes ethanol or propanol. The gel includes jelly. The jelly includes aloe vera jelly. The hair glue further includes flavoring agent. The photochromic material can change color when exposed to sunlight.

### BRIEF DESCRIPTION OF THE DRAWINGS

The aforementioned aspects and the advantages of the present invention may be readily understood by the following detailed descriptions, and the spirit of the present invention may be easily understood by the descriptions and the attached drawings below, wherein:

FIG. 1 illustrates a diagram of one embodiment of the present invention.

### DETAILED DESCRIPTION OF THE PREFERRED EMBODIMENT

In the following descriptions, various specific details are provided for comprehensive understanding of the embodiments of the present invention. The present invention will now be described in detail with the preferred embodiments and the attached drawings, and it should be comprehended that all of the preferred embodiments of the present invention are provided only for illustrating but not for limiting the Claims of the present invention. It should also be understood by the person having ordinary skill in the art that the present invention may be performed without one or more specific details or other specific methods. Generally, a hair glue has strong setting ability, and can be made into spray type, smear type, etc. The composition of the hair glue mainly includes film-forming agents, conditioning agents, diluents and other additives, etc. The percentage of each agent may be different in use, so as to form different effects of setting, beautification and maintenance. The main function of the hair glue is to set the hair style, and the main composition of the hair glue includes gel, for example Polyvinyl Chloride (PVC), and solvent, for instance ethanol. There may be butane (one type of hydrocarbon) in the hair glue. In one embodiment, the hair glue may include rosin, acrylic resin alkanolamine solution, ethanol, flavoring agent, and so on. It is a liquid or gel which is transparent and has adhesiveness depending on whether it is made into spray type or cream type.

The present invention can be employed in the following products: hair spray, a compressed gas containing a great amount of alcohol and chemicals and usually diffusively sprayed on the hair; hair mousse; hair gel, mixed by alcohol and jelly. The alcohol quickly evaporates and the jelly remains on the hair evenly when the hair gel is exposed to air. In another embodiment, the main composition of the hair glue includes aloe vera jelly, moisture (or pure water), alcohol (for example propanediol, glycerol), trophoblast protein, flavoring agent. When used, the hair glue is sprayed on the desired position of the hair. The hair glue mainly achieves setting function, and some products can also achieve the functions of moisture retention, lightening hair radiance and maintenance.

The hair glue of the present invention includes thermochromic or photochromic material doped therein. The photochromic or thermochromic materials are evenly dispersed in the gel. In addition to that the dye may be doped therein, microcapsule technology can be utilized to manufacture the hair glue. A thermochromic material can change color due to a heat change. When the thermochromic material is not subject to the heat or temperature, it can turn back to the original color. A thermochromic material can change color repeatedly due to a change in temperature. In addition to the photochromic or thermochromic material, the hair glue may also include leuco dye, color forming agent and temperature control agent. Different temperature control factors may be employed to generate different color change intervals. The method mentioned above may be utilized to manufacture a discolored hair glue. A photochromic material can change color when exposed to sunlight or ultraviolet light. When the photochromic material is not subject to the sunlight or ultraviolet light, it can turn back to the original color. The photochromic or thermochromic material may be optionally doped with leuco dye, light stabilizer and ultraviolet absorber in the gel. The photochromic or thermochromic material can absorb sunlight or UV light to change its structure, so as to change its color and achieve the effect of changing the color of the hair. The present invention can employ photochromic dye doping. The photochromic dye may be spiropyrans, spiroxazines, fulgide, fulgimides, benzopyran, naphthopyran, spirobenzopyran, spironaphthopyran, spirobenzoxazine or spironaphthoxazine. The photochromic or thermochromic material may be employed or the discolored molecules may be added into the gel to render the whole matrix to be dispersed with the discolored molecules evenly; the color may be modulated from pale color to dark color based on different wavelengths. Microcapsule technology can be utilized to make color modulation, so as to change temperature based on the exposure to light and change color based on the heat change. When it is not subject to the heat or temperature, it can turn back to the original color. The photochromic or thermochromic material can generate different color change intervals by utilizing different temperature control factors. Adding antioxidants or/and ultraviolet absorber can enhance the anti-light fatigue. The photochromic or thermochromic material may be organic dye and can achieve the function of complete light transmission.

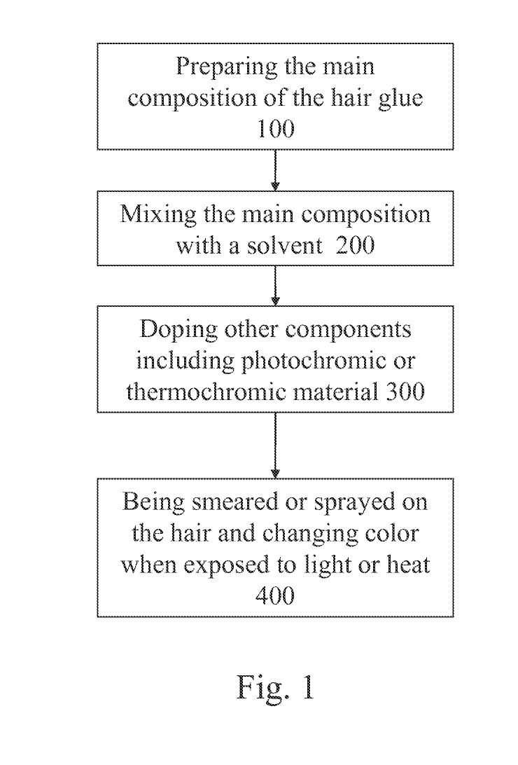

The method thereof may refer to FIG. 1. In step 100, the main composition of of the hair glue, for instance gel, is prepared. In step 200, the main composition is mixed with a solvent. Then, the mixture is doped with other components including the photochromic or thermochromic material in step 300. Subsequently, the completed composition is smeared or sprayed on the hair and changes color when exposed to light or heat in step 400. The step 200 can be exchanged with the step 300, or may be performed with the step 300 at the same time.

The present invention is described above with the preferred embodiments. However, it should be appreciated that this embodiment is described not for limiting the Claims of the present invention. Numerous alterations and modifications may be practiced by those skilled in the art without departing from the spirit and scope of the present invention. It is intended that all such modifications and alterations are included insofar as they come within the scope of the present invention as claimed or the equivalents thereof.

What is claimed is:

1. A hair glue comprising: gel, solvent, and photochromic or thermochromic material doped therein to allow hair to change color.

2. The hair glue of claim 1, wherein said gel comprises temperature control agent, light stabilizer, ultraviolet absorber or the combination thereof.

3. The hair glue of claim 2, wherein said solvent comprises alcohol.

4. The hair glue of claim 3, wherein said alcohol comprises ethanol or propanol.

5. The hair glue of claim 3, further comprising water.

6. The hair glue of claim 3, wherein said gel comprises jelly.

7. The hair glue of claim 6, wherein said jelly comprises aloe vera jelly.

8. The hair glue of claim 1, wherein said solvent comprises alcohol.

9. The hair glue of claim 8, wherein said alcohol comprises ethanol or propanol.

10. The hair glue of claim 8, further comprising water.

11. The hair glue of claim 8, wherein said gel comprises jelly.

12. The hair glue of claim 11, wherein said jelly comprises aloe vera jelly.

13. The hair glue of claim 1, further comprising flavoring agent.

14. The hair glue of claim 1, wherein said photochromic material changes color when exposed to sunlight.

15. The hair glue of claim 1, wherein said thermochromic material changes color due to a change in temperature.
